(19) United States
(12) Patent Application Publication
(10) Pub. No.: US 2024/0017082 A1
(43) Pub. Date: Jan. 18, 2024

(54) CLASSICAL CONDITIONING OF NEUROMODULATION WITH SYNCHRONIZED MUSICAL STIMULATION

(71) Applicant: The Florida State University Research Foundation, Inc., Tallahassee, FL (US)

(72) Inventors: Kevin Johnson, Tallahassee, FL (US); Frank Andrew Kozel, Tallahassee, FL (US); Jayne Standley, Tallahassee, FL (US)

(21) Appl. No.: 18/222,114

(22) Filed: Jul. 14, 2023

Related U.S. Application Data
(60) Provisional application No. 63/389,424, filed on Jul. 15, 2022.

(51) Int. Cl. A61N 2/00; A61B 5/395; A61N 2/02; G10H 1/00

(52) U.S. Cl. CPC A61N 2/002; A61N 2/006; A61B 5/395; A61N 2/008; A61N 2/02; G10H 1/0025; G10H 2210/111

(57) ABSTRACT

A device may include a transcranial magnetic stimulation (TMS) device configured to generate a localized magnetic field waveform at a brain region. A device may include an acoustic controller configured to generate an audio waveform having at least one of rhythm, harmonic element, or melodic element synchronized to the localized magnetic field waveform. In various implementations, the brain region is a motor cortex of a patient. The device can evoke or produce neurostimulation effects through acoustic stimulation (e.g., music) through classical (or Pavlovian) conditioning paired in combination with synchronized transcranial magnetic stimulation output. The device can be used to extend the effects of neuromodulation, thus reducing the burden of in-patient visits (for procedural neurostimulation), reducing battery life demand of implanted devices, or simply improving the patient experience with achieving benefits (i.e., by listening to music versus unnatural sensory sensations with neurostimulation).

### CROSS-REFERENCE TO RELATED APPLICATIONS

This application claims priority to U.S. Provisional Application Ser. No. 63/389,424 filed Jul. 15, 2022, the disclosure of which is expressly incorporated herein by reference.

### BACKGROUND

Transcranial Magnetic Stimulation (TMS) is a non-invasive neuromodulation treatment method and an FDA-cleared approach to stimulate the brain for treating depression, OCD, and smoking cessation (with many other clinical applications under investigation). A TMS system includes an electric pulse generator or stimulator that is connected to a magnetic coil that is connected to the scalp to generate a varying magnetic field via electromagnetic induction to cause an electric current at a specific area of the brain. TMS over the motor cortex, at sufficient machine power, can cause a thumb/hand movement.

TMS treatments are nevertheless cumbersome to perform and are performed before trained technicians in a doctor's office or clinic.

### SUMMARY

An exemplary system and method are described that can evoke or produce neurostimulation effects through acoustic stimulation (e.g., music) through classical (or Pavlovian) conditioning paired in combination with synchronized transcranial magnetic stimulation output. The exemplary system can be used to extend the effects of neuromodulation, thus reducing the burden of in-patient visits (for procedural neurostimulation), reducing battery life demand of implanted devices, or simply improving the patient experience with achieving benefits (i.e., by listening to music versus unnatural sensory sensations with neurostimulation).

The term “synchronized” refers to the acoustic stimulation (e.g., music) having a tone, musical tempo, rhythm, or acoustic pattern that is matching or common to that of the frequency of transcranial magnetic stimulation. For example, transcranial magnetic stimulation may have a stimulation sequence comprising a frequency of stimulation between 1 Hz and 10 Hz. Transcranial magnetic stimulation may also be applied at higher frequency of up to 18 Hz or at bursts of up to 50 Hz (higher than music tempo).

The exemplary system and method can be applied to devices for neuromodulation, both invasive (e.g., deep brain stimulation, spinal cord stimulation, vagus nerve stimulation, etc.) and non-invasive (transcranial magnetic stimulation, transcranial electrical nerve stimulation, peripheral nerve stimulators, etc.). This system can be applied to enhance all such devices but is preferable to non-invasive approaches that require an extensive number of in-patient visits (such as Transcranial Magnetic Stimulation, which currently requires 30+ visits over six weeks).

In classical conditioning, the Unconditioned Stimulus (US) inherently produces an Unconditioned Response (UR); when the US is paired with a Conditioning Stimulus (CS), the CS alone subsequently produces a Conditioned Response (CR) that is similar to the UR.

Musical rhythm can inspire movement and likely modulates motor cortex excitability. By synchronizing the beat of musical rhythm with TMS pulses over the motor cortex, the system can generate a complementary effect for pain reduction. In some embodiments, the exemplary system and method can be used to improve symptoms of individuals with limb pain. Movement and the motor system are tightly linked to the pain experience, and activating the motor system can have therapeutic benefits. Musical rhythms can inspire movement and evoke activity of the motor cortex [1-3]. However, passive music listening does not reliably induce movement, and active exercise yields variable results due to individual abilities. TMS is a non-invasive method to stimulate the motor cortex with precise timing, and TMS can generate movement in targeted muscle groups. A study has previously shown that an auditory tone can be paired with single TMS pulses to impact the motor cortex [4]. By pairing meaningful musical rhythms in sync with TMS pulses, the exemplary system and method can employ the entrainment or association to enhance the neuromodulation effect of music on the motor cortex.

Indeed, the exemplary system and method, by pairing neurostimulation devices (US) with music (CS), can be used for the subsequent presentation of music alone. Neurostimulation may be achieved through invasive devices (e.g., peripheral nerve, spinal cord or brain stimulators) or non-invasive devices (e.g., transcutaneous nerve stimulators, transcranial brain stimulation such as electrical or Transcranial Magnetic Stimulation). Music elements may include speakers for audio output, instruments, somatic or tactile sensory outputs, or visual experiences. Music alone may be initiated “on-demand,” or on a time or event-based schedule.

In an aspect, a system is disclosed comprising a transcranial magnetic stimulation (TMS) device configured to generate a localized magnetic field waveform at a brain region (e.g., cortex region of the brain); and an acoustic controller configured to generate an audio waveform having at least one of rhythm, harmonic element, or melodic element synchronized to the localized magnetic field waveform.

In some embodiments, the system further includes a TMS controller, wherein the TMS controller is configured to generate a TMS waveform from a TMS waveform file, wherein the TMS waveform is amplified and subsequently applied to one or more TMS coils to generate the localized magnetic field waveform.

In some embodiments, the TMS controller is configured to adjust a phase offset of the TMS waveform to synchronize the TMS waveform with the audio waveform.

In some embodiments, the acoustic controller is configured to adjust a phase offset of the audio waveform to synchronize the audio waveform with the TMS waveform.

In some embodiments, the system further includes a piece of electromyographic equipment configured to acquire electromyography (EMG) during operations of the transcranial magnetic stimulation and acoustic controller.

In another aspect, a method is disclosed to perform a transcranial magnetic stimulation treatment, the method comprising generating, via a transcranial magnetic stimulation (TMS) device, a localized magnetic field stimulation at a brain region (e.g., cortex region of the brain) of a patient; and concurrent with the localized magnetic field waveform, generating, via an acoustic controller, an acoustic stimulation having at least one of rhythm, harmonic element, or melodic element synchronized to the localized magnetic field stimulation.

In some embodiments, the method further includes repeating the localized magnetic field stimulation and the concurrent and synchronized acoustic stimulation over a plurality of treatment sessions to establish entrainment or conditioning, wherein the acoustic stimulation can be subsequently performed to invoke a conditioned response or entrainment response without presence of the localized magnetic field stimulation.

In some embodiments, the localized magnetic field stimulation is used to treat pain.

In some embodiments, the localized magnetic field stimulation is used to treat chronic pain.

In some embodiments, the method further includes receiving, via the TMS device, a TMS file; receiving, via the acoustic controller, an audio file; and adjusting a phase offset of a TMS waveform generated from the TMS file to synchronize the TMS waveform with the audio waveform, wherein the TMS waveform is amplified and subsequently applied to one or more TMS coils to generate the localized magnetic field waveform.

In some embodiments, the method further includes receiving, via the TMS device, a TMS file; receiving, via the acoustic controller, an audio file; and adjusting a phase offset of the audio waveform generated from the audio file to synchronize the audio waveform to the TMS waveform, wherein the TMS waveform is amplified and subsequently applied to one or more TMS coils to generate the localized magnetic field waveform.

In some embodiments, the method further includes acquiring an electromyography (EMG) measurement during operations of the generation of the localized magnetic field waveform and the acoustic stimulation.

In some embodiments, execution of the instructions by a processor causes the processor to cause, a transcranial magnetic stimulation (TMS) device, to generate a localized magnetic field waveform at a brain region (e.g., cortex region of the brain); and cause, an acoustic controller, to generate an audio waveform having at least one of rhythm, harmonic element, or melodic element synchronized to the localized magnetic field waveform.

In some embodiments, the instructions further cause the processor to receive a TMS waveform file; and generate a TMS waveform to be amplified and subsequently applied to one or more TMS coils to generate the localized magnetic field waveform.

In some embodiments, the instructions further cause the processor to adjust a phase offset of the TMS waveform to synchronize the TMS waveform with the audio waveform.

In some embodiments, the instructions further cause the processor to adjust a phase offset of the audio waveform to synchronize the audio waveform with the TMS waveform.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIGS. 1A-1C illustrates an exemplary alignments of musical beats to transcranial magnetic stimulation (TMS) frequencies according to various aspects of the disclosure.

FIG. 2 illustrates an overview of an exemplary configuration for experimental setup to evaluate the exemplary system and method according to various aspects of the disclosure.

FIG. 3A illustrates an example TMS-MUSIC system comprising a TMS system and associated acoustic/music system according to various aspects of the disclosure.

FIG. 3B illustrates an example acoustic waveform that may be generated in combination with the TMS stimulation waveform according to various aspects of the disclosure.

FIG. 4 illustrates an example method for a study to evaluate the efficacy of music synchronized in combination with TMS stimulation.

FIG. 5 illustrates an exemplary computer system suitable for implementing the several aspects of the disclosure.

### DETAILED DESCRIPTION

Each and every feature described herein, and each and every combination of two or more of such features, is included within the scope of the present invention provided that the features included in such a combination are not mutually inconsistent.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

Pain and the Motor System

Pain and movement are directly coupled, and thus the motor cortex is a therapeutic target for treating pain [5]. At the level of the spinal cord, nociceptive reflexes can trigger a rapid movement away from a noxious or harmful stimulus. The close link of these systems also occurs in the brain [6], and divergent research points to possible changes in the motor cortex with chronic pain [7, 8]. Implanted stimulators over the motor cortex are used clinically to treat severe chronic pain, and non-invasive stimulation methods have a growing body of evidence to support potential efficacy [5, 9, 10]. A study has previously demonstrated the potential of Transcranial Magnetic Stimulation (TMS) over the motor cortex for a rare form of severe limb pain [11], and a recent TMS consensus review supports motor cortex targeting for neuropathic pain [9].

Fear-avoidance and movement behavior likely contribute to the transition from acute injury to chronic musculoskeletal pain [12, 13]. Guarding and rest are adaptive responses to acute pain; however, restoration of movement is a key component of healing. Movement-based interventions may be an important component for regaining function and preventing prolonged disability [14, 15]. Fear of pain with movement is often a barrier to recovery, and combinations of psychological and physical therapies can help patients reduce the daily interference caused by chronic pain [15].

Pain is a complex sensory and emotional experience that impacts thoughts and behaviors. While there are many biological targets and psychological components of pain, the motor system and movement correspond to sensory and behavioral aspects. Objective measures of motor cortex activity are established and readily accessible. The exemplary system and method may be employed for neuromodulation of the motor cortex as well as for other brain processes involving the pain experience.

Musical Rhythm Effects on the Motor System

Music is an ancient form of neuromodulation, and musical rhythm can both originate from and also inspire movement [16]. Musical rhythm can alter the excitability of the motor cortex, as measured by motor-evoked potentials [1-3]. TMS is a valuable neuroscience tool to measure cortical excitability using measures of muscle activity such as electromyography (EMG) or visually observed motor thresholds. In particular, TMS is a non-invasive way to stimulate the motor cortex, and TMS over the motor cortex can cause movement in corresponding limbs (e.g., stimulation of the left motor cortex thumb region will cause the right thumb to move). Increases in TMS-induced motor-evoked potentials indicate cortical excitability, while decreases indicate cortical inhibition. Certain musical rhythms can induce the urge to move [17, 18] and likely increase cortical excitability [2, 3].

Musical rhythm has been used therapeutically to modulate movement, primarily for movement disorders [19-22]. There are examples of music used for pain management, although the focus is often on attentional or emotional aspects during acute pain (e.g., distraction, reducing anxiety) [23-28]. The exemplary system and method may employ rhythm focused on the modulation of movement for pain and may further include musical components for capturing attention and improving mood. There are some potential limitations to musical rhythm in therapy. Passive music listening is often not sufficient to evoke physical movement. Active music therapy may be dependent on rhythm preferences, musical abilities to perceive and produce rhythms, and even the body part moving (e.g., head vs. hand) [16, 29]. The exemplary system and method may bypass such dependencies by building associations between the perception of musical rhythm with the direct stimulation of the motor cortex to produce movement.

Transcranial Magnetic Stimulation Effect on the Motor System

TMS can be used as a neuroscience tool for measuring brain function as well as a research and clinical tool to change brain function [30, 31]. TMS as a neuroscience tool uses single pulses to probe excitability, whereas repetitive stimulation with multiple pulses (e.g., 300-3000 pulses) can drive a lasting change in brain function. The frequencies or patterns of stimulation can have either inhibitory or excitatory effects (e.g., slow 1 Hz is considered to be inhibitory while faster 10 Hz is reported to be excitatory) [31]. These effects can translate into therapeutic benefits. TMS is FDA cleared for treating depression at 10 Hz when placed over the prefrontal cortex. Multiple studies have been piloted for repetitive TMS for treating severe chronic limb pain. It has been observed that there are visual signs of increased motor excitability and remarkable symptom improvement for many patients [11]. The exemplary system and method in combining musical rhythm with repetitive TMS enhance the cumulative excitatory effect.

Combined Effect of Musical Rhythm and TMS to Modulate the Motor Cortex

Musical rhythm and TMS may operate synergistically to create effects beyond either alone. In some embodiments, the TMS-MUSIC system may employ musical rhythm as the foundation and be used to enhance the induction of movement from musical rhythm by adding synchronized TMS. The immediate effect is expected to be stronger than passive music listening (which may not evoke movement) and even active music engagement (given the precise timing, especially for those with limited rhythm tracking abilities).

It is expected that synchronized rhythm with TMS will produce greater cortical excitability than the musical rhythm alone (with sham TMS). It is also expected that the effect of TMS on the motor cortex to be amplified by musical rhythm. When an individual imagines movement (without actually moving), TMS subsequently evokes a stronger movement as the motor cortex is primed for activity [32, 33]. It is thus anticipated that musical rhythm that inspires movement will likewise result in stronger evoked movement with TMS.

In addition to the combined effects of simultaneous stimulation, there may also be associative learning or entrainment from pairing musical rhythm and TMS. A study was previously conducted that investigated Pavlovian (classical) conditioning applied to TMS [4]. Specifically, the study paired an auditory tone with a TMS pulse, which subsequently led to the tone alone evoking a motor cortex response. Musical rhythm is likely a superior stimulus for conditioning, as a musical beat can inherently motivate movement (whereas an auditory tone is neutral or may even freeze movement). It is expected that pairing musical rhythm and TMS leads can lead to more spontaneous motor-evoked potentials in response to musical beats alone during in-office sessions. At-home listening may extend the effects, given the developed association between musical rhythm and movement. The exemplary system and method may be used to enhance and entrain musical rhythm for use beyond in-office settings.

### Example Method

Practical innovation occurs at the intersection of 1) unmet needs, 2) emerging science and technology, and 3) a sustainable solution. The need for safe and effective pain management tools is well-known [34]; however, such tools should have durable effects and/or fit into the daily life of an individual with chronic pain. Music therapies can be readily accessible, and the exemplary system and method seek to enhance effects for subsequent durable or daily use. The exemplary system and method may be used to build the science of a synergistic approach to modulate the motor cortex for pain reduction and to establish parameters that could inform a pivotal clinical trial, e.g., and employed as a sustainable solution. TMS is routinely used in a large number of clinics, with insurance reimbursement for major depressive disorder. Synchronized music with TMS is disclosed herein, and there are conditions beyond the pain that may also benefit from a synchronized musical TMS approach (including depression, mood disorders, movement disorders, brain-based disorders). other conditions (e.g. depression,)

### Example System

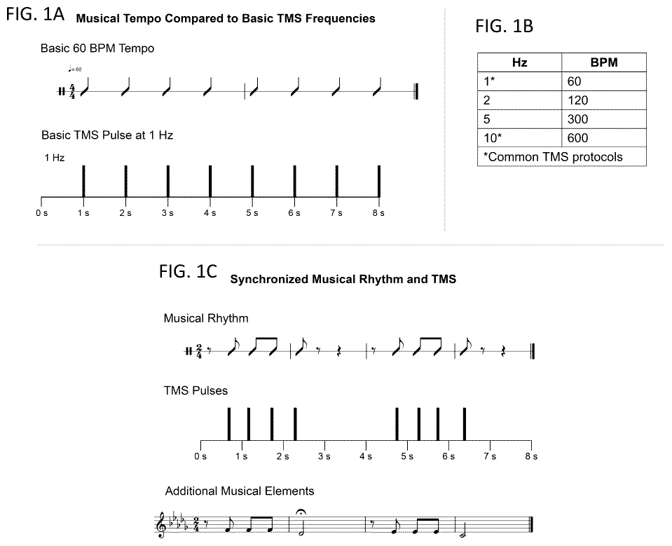

FIGS. 1A-1C illustrates an exemplary alignments of musical beats to transcranial magnetic stimulation (TMS) frequencies according to various aspects of the disclosure. FIG. 1A shows a relatively slow tempo/rhythm (60 BPM) aligned to a slow, inhibitory TMS frequency (1 Hz). FIG. 1B shows a table of common, established TMS frequencies correspond to musical tempos ranging from relatively slow (e.g., 1 Hz and 60 BPM) to quite fast (e.g., 10 Hz and 600 BPM). FIG. 1C shows a musical rhythm can have TMS pulses delivered in sync. Additional musical elements can be added to enhance the overall experience.

FIGS. 1A-1C provide an overview of the conceptual framework of this disclosure. Both musical tempo (beats) and basic TMS frequencies (pulses) can be made to occur simultaneously. Advancing one step further, musical rhythms can be combined with the TMS pulses to create novel stimulation patterns. Given the effect of musical rhythm on the motor cortex, one might expect these novel “musical TMS” patterns to have unique properties beyond basic frequencies. Even further, additional musical elements can be layered to utilize the multi-component therapeutic aspects of music (e.g., capture attention, motivate, modulate mood states, etc.).

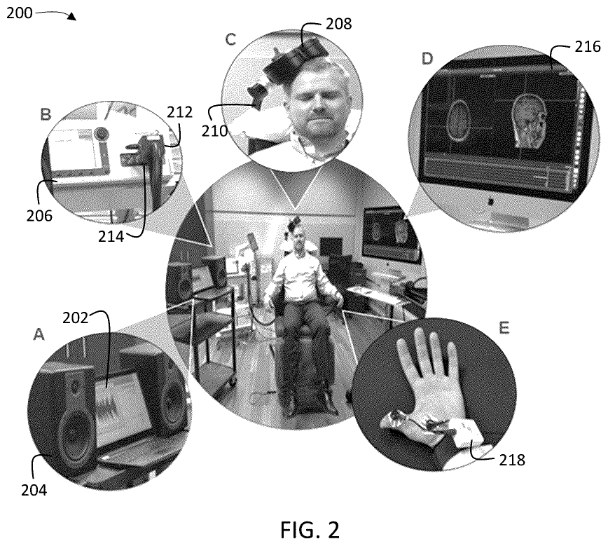

FIG. 2 provides an overview of a configuration 200 for experimental setup to evaluate the exemplary system and methods disclosed herein. The configuration 200 includes audio system 202 for supplying audio data, including musical tempo, rhythms, and/or other musical elements. The configuration 200 also includes speakers 204 that play the audio data supplied by the audio system 202. The configuration 200 also includes a transcranial magnetic stimulator 206 (e.g., power and controller unit) configured to supply TMS frequencies (pulses). The configuration 200 also includes a TMS coil 208 coupled to the transcranial magnetic stimulator 206 via an electrical cable 210 with a connector 212. The connector 212 is configured to be removably coupled to a socket 214 of the transcranial magnetic stimulator 206. In some implementations, the electrical cable 210 may be an integral component of the transcranial magnetic stimulator 206 such that it is not removable. In such instances, the connector 212 and socket 214 may be omitted. The configuration 200 may also include a neuronavigation system 216 to ensure accurate positioning of TMS pulses. In some implementations, the configuration 200 may also include an electromyography (EMG) 218 to quantify electrical activity in muscle tissue of the contralateral limb.

The audio system 202 may use software (e.g., Matlab) to play audio data (e.g., basic rhythms) via the speakers 204 and send trigger commands to the transcranial magnetic stimulator 206. As described above, the trigger commands may be synchronized to the audio data. The audio system 202 may also use musical software and MIDI hardware to enhance flexibility for music creation and playback of more complex audio data. In order to have strong safety controls governing transcranial magnetic stimulator 206, the exemplary transcranial magnetic stimulator 206 may include TMS device software to output signals to control the pacing of the musical rhythm of audio system 202. Therefore, bidirectional control communications may be exchanged between the audio system 202 and the transcranial magnetic stimulator 206. The configuration 200 facilitates placement of the TMS coil 208 over the motor cortex of a patient. The neuronavigation system 216 may be used to ensure accurate positioning despite any potential head or coil movement. The non-invasive stimulation creates visible movement in the contralateral limb, which can be objectively quantified using the EMG 218.

### Additional Example Systems

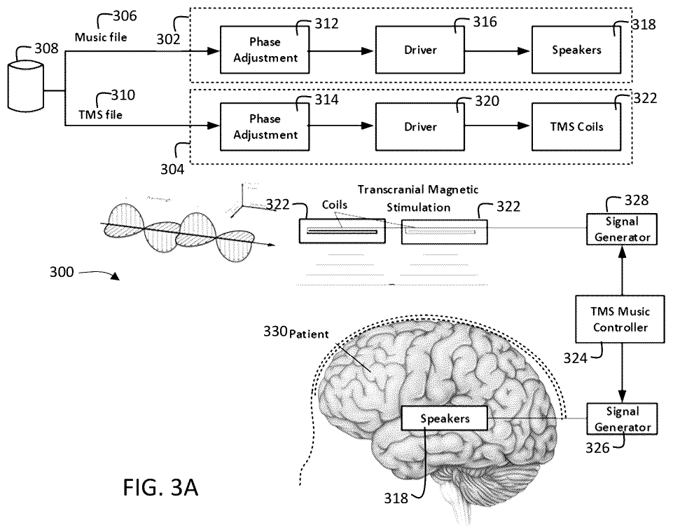

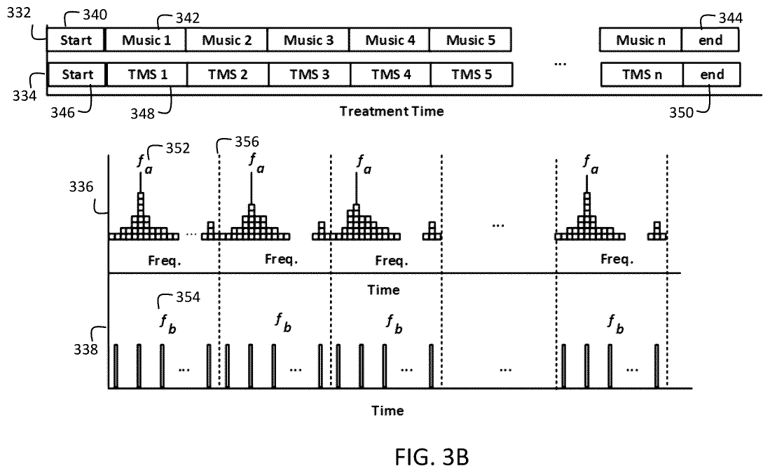

FIG. 3A shows an example TMS-MUSIC system 300 comprising a TMS system 302 and associated acoustic system 304. The acoustic system 304 is configured to receive a music file 306 from a database 308. The music file 306 may be a midi file, MP3 file, or the like, in which the audio or music has tempo/rhythm, harmonic element, or melodic elements synchronized to a TMS stimulation file 310. The TMS stimulation file 310 includes timed pulses for generating desired transcranial magnetic stimulation from one or more coils.

A melodic element is a linear succession of musical tones that a listener perceives as a single entity. Harmony refers to a set of notes that are joined together or composed into whole units or compositions. Tempo refers to the speed or pace of the musical tones. Rhythm generally means a movement marked by the regulated succession of strong and weak elements or of opposite or different conditions. This general meaning of regular recurrence or pattern in time can apply to a wide variety of cyclical natural phenomena having a periodicity or frequency of anything from microseconds to several seconds.

An information flow of the TMS system 302 and the music system 304 show how the music file 306 and the TMS stimulation file 310 are processed, respectively. In the example shown, the music system 302 system includes a phase adjustment module 312 that can adjust the phase or other audio characteristics (e.g., frequency, amplitude, playback speed, etc.) of the audio/music file 306 to align to the TMS pulses in the TMS stimulation file 310. In some implementations, the TMS system 304 additionally or alternatively includes a phase adjustment module 314 that can adjust the phase or other signal characteristic of the TMS pulses in the TMS stimulation file 310 to align to audio characteristics of the music file 306. The music system 302 employs the (optionally phase adjusted) music file 306 to generate, via a driver 316 and speakers 318, the acoustic stimulation. Likewise, the TMS system 302 employs the (optionally phase adjusted) TMS stimulation file 310 to generate, via a TMS driver 320 and TMS coil 322, the magnetic field stimulation.

In an example shown in FIG. 3A, the TMS-MUSIC system 300 includes a TMS music controller 324, acoustic signal generator 326, speakers 318, a TMS signal generator 328, and one or more TMS coils 322 (two in the example shown, though three or more coils may be used). The TMS music controller 324 may process the music file 306 and the TMS stimulation file 310 as described above to generate signals to provide to the acoustic signal generator 326, the speakers 318, the TMS signal generator 328, and the TMS coils 322 for therapeutic effect of a patient 330. In some embodiments, the acoustic signal generator 326 and speakers 318 may be integrated into the TMS music controller 324. In other embodiments, multiple drivers may be employed.

FIG. 3B shows an example of an acoustic file structure 332 of the music file 306 and a TMS file structure 334 of the TMS stimulation file 310. FIG. 3B also shows an acoustic waveform 336 that may be generated based on the music file 306 in combination with a TMS stimulation waveform 338 that may be generated based on the TMS stimulation file 310.

In the example shown, the acoustic file structure 332 includes a start block 340 followed by a plurality of music segments 342 and ending with an end block 344. Each of the plurality of music segments 342 may be the same duration. The same or different audio information may be carried in each of the plurality of music segments 342. Likewise, the TMS file structure 334 includes a start block 346 followed by a plurality of TMS stimulation blocks 348 and ending with an end block 350. Each of the plurality of TMS stimulation blocks 348 may be the same duration. The same or different TMS pulse patterns may be carried in each of the plurality of TMS stimulation blocks 348. As described above, the plurality of music segments 342 may be synchronized with the plurality of TMS stimulation blocks 348.

In the example shown in FIG. 3B, the acoustic waveform 336 has a set of one or more higher frequency components 352 (shown as fa) that appears on a periodic basis 354 (shown as fb) while the TMS stimulation is being outputted. Different ones of the plurality of music segments 342 and the plurality of TMS stimulation blocks 348 are shown by the dashed divider line 356. In some implementations, each of the segments 342, 348 occur on the periodic basis 354.

### Experimental Results and Additional Examples

A study may be conducted to evaluate the efficacy of music synchronized in combination with TMS stimulation with respect to a particular clinical treatment. The study may evaluate the efficacy of subsequent music stimulation in between TMS stimulation sessions.

A goal of the study may be to optimize the rhythm for movement induction safely in sync with TMS.

Overview: Throughout both phases (R61/R33), the study may build and maintain a registry of community volunteers with chronic limb pain. Patient input may help guide the selection of rhythms, as well as refine the type of cases that may benefit from our approach. In the R61 phase, the study may create an optimized synchronized musical rhythm and TMS composition. This creation may be guided by a “target product profile” that characterizes the desired attributes. Ultimately, the study may test whether TMS enhances the effect of musical rhythm on the motor cortex (Go/No-Go criteria). If the R61 passes the Go/No-Go criteria, the study may advance to the R33 clinical trial. The trial will test if our rhythmic TMS composition has an impact on pain over 2 weeks of in-office use, and we will also explore whether any durable benefit occurs with an additional 2 weeks of only musical rhythm.

R61 and R33: Community Registry and Survey of Patients with Limb Pain

The study may build a community registry of patients with limb pain, as well as healthy volunteers as needed for the R61 Go/No-Go study and subsequent studies and future activities. The study may use social media advertisements and partnerships to recruit relevant individuals to the registry. Potential participants may complete a quick REDCap survey including basic demographics; pain characteristics (location, intensity, duration, life impact); musical rhythm perspectives; and interest in participating in our studies. Interested individuals will be invited to participate in the R61 studies. The study may use participant and patient input to guide our selections of musical rhythms in R61 formative studies, as well as refine the inclusion/exclusion criteria for the R33 study.

R61: Build Musical Rhythm Composition and the Synchronized “Target Product Profile”

The study may use the “target product profile” approach to define the desired set of attributes for the synchronized musical rhythm and TMS. While the profile can be iteratively adjusted with new information such as desired features (or reduced based on feasibility), core elements of safety and efficacy must remain. The profile also provides a checklist to be validated (based on technical knowledge) and/or verified (based on in-human testing).

Evaluation of the Combined Effect of Synchronized Rhythm and TMS Together

Extending the single session of the R61 phase, the study may administer 10 sessions over 2 weeks. For each of the 10 sessions, the study may acquire EMG from a series of equivalent TMS test pulses delivered before and after each ˜15-minute intervention. The study may estimate the independent and combined effects of synchronized rhythm and TMS and may test for replication of our R61 phase Go/No Go study.

Increases in cortical excitability may correlate with improvements in pain measures. The R33 study may deploy the TMS-music system in a population with pain to provide an estimation of the relationship between cortical excitability and pain relief. The study may evaluate effects of single-session changes with same-session pain intensity change (PROMIS Pain Intensity Scale). It is expected that consistent multi-session changes would correlate with improvements of pain interference with life activities (PROMIS Pain Interference Scale).

The Patient Selection Criteria for the R33 Study Will be Refined During the R61 Phase.

The study may require a pain condition(s) to be safe and feasible for the desired procedures. For instance, extreme limb trauma that impacts nerve conduction and muscle movement may not be feasible for EMG measures. The study may require a pain condition that is relatively stable for the duration of the protocol. For instance, acute sprains or uncomplicated post-surgical pain typically improve quickly, and pain measures over 2 weeks would be confounded by natural recovery.

The most likely condition of interest is either hand arthritis or mild carpal tunnel syndrome. The study may consider lower limb pain, especially foot pain. The last criterion may be based on recruitment feasibility, which the study may validate in the R61 phase.

Factorial Study Design

Sample size and power calculation: The study may use a 2×2 factorial design in which a total of 80 participants may be randomly assigned to one of the four arms: 1) real TMS with synchronized rhythm; 2) sham TMS with synchronized rhythm; 3) real TMS with non-synchronized rhythm; and 4) sham TMS with non-synchronized rhythm. The study may conduct six hypothesis tests for the main outcomes (e.g., pain measures) at a significance level α=0.008 after a Bonferroni correction.

R33: Exploratory Home Trial (Extended Effects with Rhythm Only)

With the repeated rhythmic beat and TMS pulse pairing, it is expected that entrainment or conditioning may develop over time. In one exploratory analysis, the study may evaluate an increase in spontaneous movement with rhythmic beats not paired with TMS pulses (as not all beats in the active arm will be paired) and with rhythmic beats paired with sub-threshold pulses (a small percentage in the active arm). If spontaneous movement is found to increase over the two weeks of in-office synchronization, these increases could provide an understanding of the time course to establish entrainment (any arm) or conditioning (active arm of paired rhythm and TMS).

Following the two weeks of in-office treatments, all groups may continue at-home listening daily for 2 weeks (either rhythm in sync with the TMS patterns or not in sync). The study may use REDCap to assess feasibility and compliance, perceptions of home use, and daily pain measures (average pain intensity, and weekly pain interference). While the sample size is unlikely to provide a definitive result, we aim to gather feedback on home use and preliminary data to guide additional studies.

Dependent on the pain condition selected and advice from our healthcare providers, we may also encourage participants to perform limited movement exercises with at-home music listening. If so, we will also collect feedback on the patient's experience with active rhythm listening.

Data Management and Statistical Analysis

Data may be collected via REDCap (surveys and ratings) and the Brainsight system (EMG and positioning variability). All data may be de-identified and blinded for analysis. The analysis may involve blinded quality checks and adjustments (EMG filtering or outlier removal). Statistical analysis will be performed using SPSS, R, Matlab, or other appropriate software.

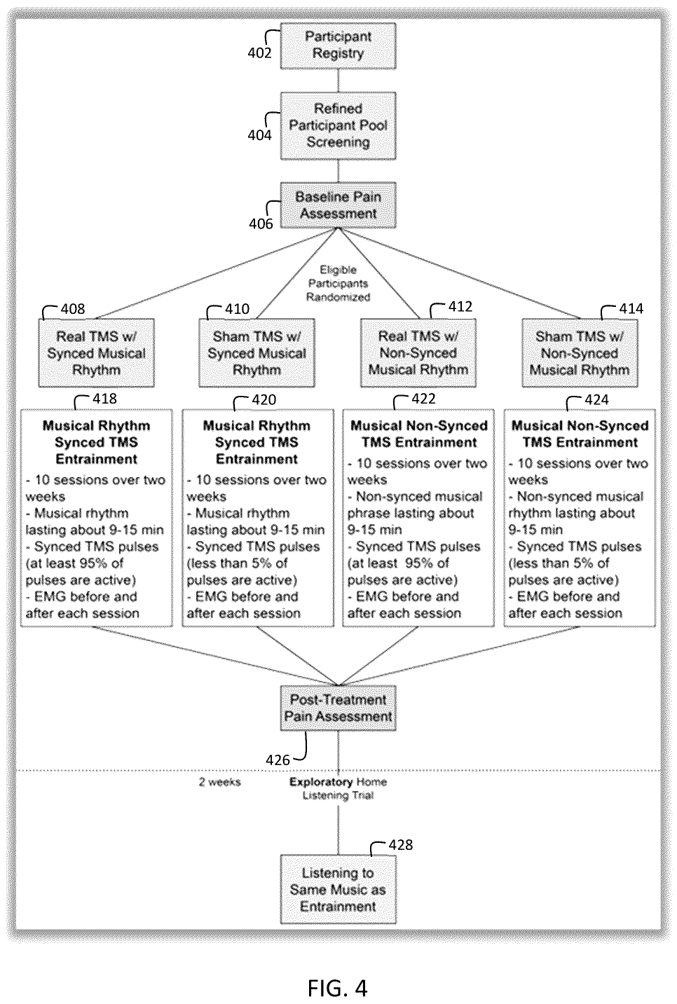

FIG. 4 illustrates an example method for a study to evaluate the efficacy of music synchronized in combination with TMS stimulation. At 402, the study may build a community registry of patients with limb pain, as well as healthy volunteers. At 404, the participant pool is screened to generate a refined participant pool. For example, the study may require a pain condition(s) to be safe, feasible, and stable for the desired procedures. At 406, a baseline pain assessment for the refined participant pool is generated to establish a starting condition of the participants.

The participants are divided into four randomized trials. At 408, real TMS pulses (e.g., active pulses or pulses designed to stimulate the motor cortex or other portion of a patient's brain) are supplied with synchronized musical rhythm. As detailed in 418, the trial includes 10 sessions over two weeks with musical rhythm lasting about 9-15 minutes being played in synchronization with TMS pulses (in some implementations, at least 95% of TMS pulses are active). An EMG is performed before and after each session.

At 410, sham TMS pulses (e.g., inactive pulses or pulses designed to not stimulate the motor cortex or other portion of a patient's brain) are supplied with synchronized musical rhythm. As detailed in 420, the trial includes 10 sessions over two weeks with musical rhythm lasting about 9-15 minutes being played in synchronization with TMS pulses (in some implementations, less than 5% of TMS pulses are active). An EMG is performed before and after each session.

At 412, real TMS pulses are supplied with non-synchronized musical rhythm. As detailed in 422, the trial includes 10 sessions over two weeks with non-synchronized musical rhythm lasting about 9-15 minutes being played with real TMS pulses (at least 95% of TMS pulses are active). An EMG is performed before and after each session.

At 414, sham TMS pulses are supplied with non-synchronized musical rhythm. As detailed in 424, the trial includes 10 sessions over two weeks with non-synchronized musical rhythm lasting about 9-15 minutes being played with sham TMS pulses (in some implementations, less than 5% of TMS pulses are active). An EMG is performed before and after each session

At 426, a post-treatment pain assessment is performed to compare with the baseline pain assessment at 406. In some implementations, at 426 it is also judged if spontaneous movement is found to increase.

At 428, all groups may continue at-home listening daily for 2 weeks (either rhythm in sync with the TMS patterns or not in sync). The study may use REDCap to assess feasibility and compliance, perceptions of home use, and daily pain measures (average pain intensity, and weekly pain interference).

### Example Computing System

It should be appreciated that the logical operations for the analysis of the MUSIC and TMS treatment described above can be implemented (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device 200 to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media.

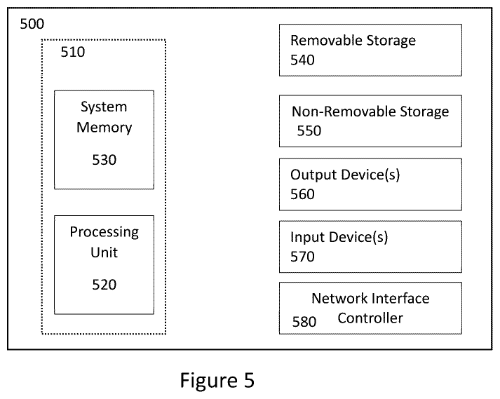

FIG. 5 illustrates an exemplary computer system 500 suitable for implementing the several embodiments of the disclosure. For example, one or more of the TMS system 302, associated acoustic system 304, or TMS music controller 324 may be implemented as the computer system 500.

It should be appreciated that the logical operations described herein with respect to the various figures may be implemented (1) as a sequence of computer implemented acts or program modules (i.e., software) running on a computing device (e.g., the computing device described in FIG. 5), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts, and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 5, an example computing device 500 upon which embodiments of the invention may be implemented is illustrated. For example, each of the TMS system 302, associated acoustic system 304, or TMS music controller 324 may be implemented as a computing device, such as computing device 500. It should be understood that the example computing device 500 is only one example of a suitable computing environment upon which embodiments of the invention may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In some embodiments, the computing device 500 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In some embodiments, virtualization software may be employed by the computing device 500 to provide the functionality of a number of servers that is not directly bound to the number of computers in the computing device 500. For example, virtualization software may provide twenty virtual servers on four physical computers. In some embodiments, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing resources hired and/or leased from a third party provider.

In its most basic configuration, computing device 500 typically includes at least one processing unit 520 and system memory 530. Depending on the exact configuration and type of computing device, system memory 530 may be volatile (such as random access memory (RAM)), non-volatile (such as read-only memory (ROM), flash memory, etc.), or some combination of the two. This most basic configuration is illustrated in FIG. 5 by dashed line 510. The processing unit 520 may be a standard programmable processor that performs arithmetic and logic operations necessary for operation of the computing device 500. While only one processing unit 520 is shown, multiple processors may be present. Thus, while instructions may be discussed as executed by a processor, the instructions may be executed simultaneously, serially, or otherwise executed by one or multiple processors. The computing device 500 may also include a bus or other communication mechanism for communicating information among various components of the computing device 500.

Computing device 500 may have additional features/functionality. For example, computing device 500 may include additional storage such as removable storage 540 and non-removable storage 550 including, but not limited to, magnetic or optical disks or tapes. Computing device 500 may also contain network connection(s) 580 that allow the device to communicate with other devices such as over the communication pathways described herein. The network connection(s) 580 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 500 may also have input device(s) 570 such as a keyboard, keypads, switches, dials, mice, track balls, touch screens, voice recognizers, card readers, paper tape readers, or other well-known input devices. Output device(s) 560 such as a printer, video monitors, liquid crystal displays (LCDs), touch screen displays, displays, speakers, etc. may also be included. The additional devices may be connected to the bus in order to facilitate communication of data among the components of the computing device 500. All these devices are well known in the art and need not be discussed at length here.

The processing unit 520 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 520 for execution. Example tangible, computer-readable media may include, but is not limited to, volatile media, non-volatile media, removable media, and non-removable media implemented in any method or technology for storage of information such as computer readable instructions, data structures, program modules or other data. System memory 530, removable storage 540, and non-removable storage 550 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

In an example implementation, the processing unit 520 may execute program code stored in the system memory 530. For example, the bus may carry data to the system memory 530, from which the processing unit 520 receives and executes instructions. The data received by the system memory 530 may optionally be stored on the removable storage 540 or the non-removable storage 550 before or after execution by the processing unit 520.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Embodiments of the methods and systems may be described herein with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method but does not exclude the presence of other compounds, materials, particles, method steps, even if the other such compounds, material, particles, method steps have the same function as what is named.

In describing example embodiments, terminology may be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document are hereby incorporated by reference in their entirety herein.
[1] Wilson E M, Davey N J. Musical beat influences corticospinal drive to ankle flexor and extensor muscles in man. Int J Psychophysiol. 2002; 44(2):177-84. doi: 10.1016/s0167-8760(01)00203-3. PubMed PMID: 11909649.
[2] Stupacher J, Hove M J, Novembre G, Schutz-Bosbach S, Keller P E. Musical groove modulates motor cortex excitability: a TMS investigation. Brain Cogn. 2013; 82(2):127-36. Epub 20130506. doi: 10.1016/j.bandc.2013.03.003. PubMed PMID: 23660433.
[3] Matthews T E, Witek M A G, Lund T, Vuust P, Penhune V B. The sensation of groove engages motor and reward networks. Neuroimage. 2020; 214:116768. Epub 20200323. doi: 10.1016/j.neuroimage.2020.116768. PubMed PMID: 32217163.
[4] Johnson K A, Baylis G C, Powell D A, Kozel F A, Miller S W, George M S. Conditioning of transcranial magnetic stimulation: evidence of sensory-induced responding and prepulse inhibition. Brain Stimul. 2010; 3(2):78-86. Epub 20090917. doi: 10.1016/j.brs.2009.08.003. PubMed PMID: 20633436.
[5] Ramos-Fresnedo A, Perez-Vega C, Domingo R A, Cheshire W P, Middlebrooks E H, Grewal S S. Motor Cortex Stimulation for Pain: A Narrative Review of Indications, Techniques, and Outcomes. Neuromodulation. 2022; 25(2):211-21. Epub 20211218. doi: 10.1016/j.neurom.2021.10.025. PubMed PMID: 35125140.
[6] Hussein A E, Esfahani D R, Moisak G I, Rzaev J A, Slavin K V. Motor Cortex Stimulation for Deafferentation Pain. Curr Pain Headache Rep. 2018; 22(6):45. Epub 20180523. doi: 10.1007/s11916-018-0697-1. PubMed PMID: 29796941.
[7] Farina S, Tinazzi M, Le Pera D, Valeriani M. Pain-related modulation of the human motor cortex. Neurol Res. 2003; 25(2):130-42. doi: 10.1179/016164103101201283. PubMed PMID: 12635511.
[8] Chang W J, O'Connell N E, Beckenkamp P R, Alhassani G, Liston M B, Schabrun S M. Altered Primary Motor Cortex Structure, Organization, and Function in Chronic Pain: A Systematic Review and Meta-Analysis. J Pain. 2018; 19(4):341-59. Epub 20180112. doi: 10.1016/j.jpain.2017.10.007. PubMed PMID: 29155209.
[9] Leung A, Shirvalkar P, Chen R, Kuluva J, Vaninetti M, Bermudes R, Poree L, Wassermann E M, Kopell B, Levy R, and the Expert Consensus P. Transcranial Magnetic Stimulation for Pain, Headache, and Comorbid Depression: INS-NANS Expert Consensus Panel Review and Recommendation. Neuromodulation. 2020; 23(3):267-90. Epub 20200325. doi: 10.1111/ner.13094. PubMed PMID: 32212288.
[10] O'Connell N E, Marston L, Spencer S, DeSouza L H, Wand B M. Non-invasive brain stimulation techniques for chronic pain. Cochrane Database Syst Rev. 2018; 4:CD008208. Epub 20180413. doi: 10.1002/14651858.CD008208.pub5. PubMed PMID: 29652088; PMCID: PMC6494527.
[11] Gaertner M, Kong J T, Scherrer K H, Foote A, Mackey S, Johnson K A. Advancing Transcranial Magnetic Stimulation Methods for Complex Regional Pain Syndrome: An Open-Label Study of Paired Theta Burst and High-Frequency Stimulation. Neuromodulation. 2018; 21(4):409-16. Epub 20180304. doi: 10.1111/ner.12760. PubMed PMID: 29504190; PMCID: PMC6033652.
[12] Edwards R R, Dworkin R H, Sullivan M D, Turk D C, Wasan A D. The Role of Psychosocial Processes in the Development and Maintenance of Chronic Pain. J Pain. 2016; 17(9 Suppl):T70-92. doi: 10.1016/j.jpain.2016.01.001. PubMed PMID: 27586832; PMCID: PMC5012303.
[13] Turk D C, Fillingim R B, Ohrbach R, Patel K V. Assessment of Psychosocial and Functional Impact of Chronic Pain. J Pain. 2016; 17(9 Suppl):T21-49. doi: 10.1016/j.jpain.2016.02.006. PubMed PMID: 27586830.
[14] Geneen L J, Moore R A, Clarke C, Martin D, Colvin L A, Smith B H. Physical activity and exercise for chronic pain in adults: an overview of Cochrane Reviews. Cochrane Database Syst Rev. 2017; 4:CD011279. Epub 20170424. doi: 10.1002/14651858.CD011279.pub3. PubMed PMID: 28436583; PMCID: PMC5461882.
[15] Booth J, Moseley G L, Schiltenwolf M, Cashin A, Davies M, Hubscher M. Exercise for chronic musculoskeletal pain: A biopsychosocial approach. Musculoskeletal Care. 2017; 15(4):413-21. Epub 20170330. doi: 10.1002/msc.1191. PubMed PMID: 28371175.
[16] Levitin D J, Grahn J A, London J. The Psychology of Music: Rhythm and Movement. Annu Rev Psychol. 2018; 69:51-75. Epub 20171016. doi: 10.1146/annurev-psych-122216-011740. PubMed PMID: 29035690.
[17] Janata P, Tomic S T, Haberman J M. Sensorimotor coupling in music and the psychology of the groove. J Exp Psychol Gen. 2012; 141(1):54-75. Epub 20110718. doi: 10.1037/a0024208. PubMed PMID: 21767048.
[18] Witek M A, Clarke E F, Wallentin M, Kringelbach M L, Vuust P. Syncopation, body-movement and pleasure in groove music. PLoS One. 2014; 9(4):e94446. Epub 20140416. doi: 10.1371/journal.pone.0094446. PubMed PMID: 24740381; PMCID: PMC3989225.
[19] Devlin K, Alshaikh J T, Pantelyat A. Music Therapy and Music-Based Interventions for Movement Disorders. Curr Neurol Neurosci Rep. 2019; 19(11):83. Epub 20191113. doi: 10.1007/s11910-019-1005-0. PubMed PMID: 31720865.
[20] Thaut M H, McIntosh G C, Hoemberg V. Neurobiological foundations of neurologic music therapy: rhythmic entrainment and the motor system. Front Psychol. 2014; 5:1185. Epub 20150218. doi: 10.3389/fpsyg.2014.01185. PubMed PMID: 25774137; PMCID: PMC4344110.
[21] Braun Janzen T, Koshimori Y, Richard N M, Thaut M H. Rhythm and Music-Based Interventions in Motor Rehabilitation: Current Evidence and Future Perspectives. Front Hum Neurosci. 2021; 15:789467. Epub 20220117. doi: 10.3389/fnhum.2021.789467. PubMed PMID: 35111007; PMCID: PMC8801707.
[22] Grau-Sanchez J, Munte T F, Altenmuller E, Duarte E, Rodriguez-Fornells A. Potential benefits of music playing in stroke upper limb motor rehabilitation. Neurosci Biobehav Rev. 2020; 112:585-99. Epub 20200221. doi: 10.1016/j.neubiorev.2020.02.027. PubMed PMID: 32092314.
[23] Tolunay T, Bicici V, Tolunay H, Akkurt M O, Arslan A K, Aydogdu A, Bingol I. Rhythm and orthopedics: The effect of music therapy in cast room procedures, a prospective clinical trial. Injury. 2018; 49(3):593-8. Epub 20180207. doi: 10.1016/j.injury.2018.02.008. PubMed PMID: 29454656.
[24] Gooding L, Swezey S, Zwischenberger J B. Using music interventions in perioperative care. South Med J. 2012; 105(9):486-90. doi: 10.1097/SMJ.0b013e318264450c. PubMed PMID: 22948329.
[25] Eid M M, Abdelbasset W K, Abdelaty F M, Ali Z A. Effect of physical therapy rehabilitation program combined with music on children with lower limb burns: A twelve-week randomized controlled study. Burns. 2021; 47(5):1146-52. Epub 20201127. doi: 10.1016/j.burns.2020.11.006. PubMed PMID: 33288333.
[26] Golino A J, Leone R, Gollenberg A, Christopher C, Stanger D, Davis T_{M}, Meadows A, Zhang Z, Friesen M A. Impact of an Active Music Therapy Intervention on Intensive Care Patients. Am J Crit Care. 2019; 28(1):48-55. doi: 10.4037/ajcc2019792. PubMed PMID: 30600227.
[27] Leonard H. Live Music Therapy During Rehabilitation After Total Knee Arthroplasty: A Randomized Controlled Trial. J Music Ther. 2019; 56(1):61-89. doi: 10.1093/jmt/thy022. PubMed PMID: 30770536.
[28] Sibanda A, Carnes D, Visentin D, Cleary M. A systematic review of the use of music interventions to improve outcomes for patients undergoing hip or knee surgery. J Adv Nurs. 2019; 75(3):502-16. Epub 20181119. doi: 10.1111/jan.13860. PubMed PMID: 30230564.
[29] Michaelis K, Wiener M, Thompson J C. Passive listening to preferred motor tempo modulates corticospinal excitability. Front Hum Neurosci. 2014; 8:252. Epub 20140424. doi: 10.3389/fnhum.2014.00252. PubMed PMID: 24795607; PMCID: PMC4006054.
[30] Rossi S, Antal A, Bestmann S, Bikson M, Brewer C, Brockmoller J, Carpenter L L, Cincotta M, Chen R, Daskalakis J D, Di Lazzaro V, Fox M D, George M S, Gilbert D, Kimiskidis V K, Koch G, Ilmoniemi R J, Lefaucheur J P, Leocani L, Lisanby S H, Miniussi C, Padberg F, Pascual-Leone A, Paulus W, Peterchev A V, Quartarone A, Rotenberg A, Rothwell J, Rossini P M, Santarnecchi E, Shafi M M, Siebner H R, Ugawa Y, Wassermann E M, Zangen A, Ziemann U, Hallett M, basis of this article began with a Consensus Statement from the Ifcn Workshop on “Present FoTMSSEGSOutA. Safety and recommendations for TMS use in healthy subjects and patient populations, with updates on training, ethical and regulatory issues: Expert Guidelines. Clin Neurophysiol. 2021; 132(1):269-306. Epub 20201024. doi: 10.1016/j.clinph.2020.10.003. PubMed PMID: 33243615; PMCID: PMC9094636.
[31] Rossi S, Hallett M, Rossini P M, Pascual-Leone A, Safety of TMSCG. Safety, ethical considerations, and application guidelines for the use of transcranial magnetic stimulation in clinical practice and research. Clin Neurophysiol. 2009; 120(12):2008-39. Epub 20091014. doi: 10.1016/j.clinph.2009.08.016. PubMed PMID: 19833552; PMCID: PMC3260536.
[32] Li S, Stevens J A, Rymer W Z. Interactions between imagined movement and the initiation of voluntary movement: a TMS study. Clin Neurophysiol. 2009; 120(6):1154-60. Epub 20090227. doi: 10.1016/j.clinph.2008.12.045. PubMed PMID: 19250861; PMCID: PMC2897011.
[33] Li S. Movement-specific enhancement of corticospinal excitability at subthreshold levels during motor imagery. Exp Brain Res. 2007; 179(3):517-24. Epub 20061208. doi: 10.1007/s00221-006-0809-8. PubMed PMID: 17160400; PMCID: PMC2889909.
[34] Institute of Medicine (U.S.). Committee on Advancing Pain Research Care and Education. Relieving pain in America: a blueprint for transforming prevention, care, education, and research. Washington, D.C.: National Academies Press; 2011. xvii, 364 p. p.

What is claimed is:

1. A system comprising:
a transcranial magnetic stimulation (TMS) device configured to generate a localized magnetic field waveform at a brain region; and
an acoustic controller configured to generate an audio waveform having at least one of rhythm, harmonic element, or melodic element synchronized to the localized magnetic field waveform.

2. The system of claim 1, wherein the brain region is the motor cortex region of the brain.

3. The system of claim 1, further comprising:
a TMS controller, wherein the TMS controller is configured to generate a TMS waveform from a TMS waveform file, wherein the TMS waveform is amplified and subsequently applied to one or more TMS coils to generate the localized magnetic field waveform.

4. The system of claim 3, wherein the TMS controller is configured to adjust a phase offset of the TMS waveform to synchronize the TMS waveform with the audio waveform.

5. The system of claim 3, wherein the acoustic controller is configured to adjust a phase offset of the audio waveform to synchronize the audio waveform with the TMS waveform.

6. The system of claim 1, further comprising:
an electromyographic equipment configured to acquire an electromyography (EMG) measurement during operation of the transcranial magnetic stimulation and acoustic controller.

7. A method to perform a transcranial magnetic stimulation treatment, the method comprising:
generating, via a transcranial magnetic stimulation (TMS) device, a localized magnetic field stimulation at a brain region of a patient; and
concurrent with the localized magnetic field stimulation, generating, via an acoustic controller, an acoustic stimulation having at least one of rhythm, harmonic element, or melodic element synchronized to the localized magnetic field stimulation.

8. The method of claim 7, wherein the brain region is the motor cortex region of the brain.

9. The method of claim 7, further comprising:
repeating the localized magnetic field stimulation and the concurrent and synchronized acoustic stimulation over a plurality of treatment sessions to establish entrainment or conditioning, wherein the acoustic stimulation can be subsequently performed to invoke a conditioned response or entrainment response without presence of the localized magnetic field stimulation.

10. The method of claim 7, wherein the localized magnetic field stimulation is used to treat pain.

11. The method of claim 7, wherein the localized magnetic field stimulation is used to treat chronic pain.

12. The method of claim 9, further comprising:
receiving, via the TMS device, a TMS file;
receiving, via the acoustic controller, an audio file; and
adjusting a phase offset of a TMS waveform generated from the TMS file to synchronize the TMS waveform with the audio waveform, wherein the TMS waveform is amplified and subsequently applied to one or more TMS coils to generate the localized magnetic field waveform.

13. The method of claim 9, further comprising:
receiving, via the TMS device, a TMS file;
receiving, via the acoustic controller, an audio file; and
adjusting a phase offset of the audio waveform generated from the audio file to synchronize the audio waveform to the TMS waveform, wherein the TMS waveform is amplified and subsequently applied to one or more TMS coils to generate the localized magnetic field waveform.

14. The method of claim 9, further comprising:
acquiring an electromyography (EMG) measurement during operations of the generation of the localized magnetic field waveform and the acoustic stimulation.

15. A non-transitory computer-readable medium having instructions stored thereon, wherein execution of the instructions by a processor causes the processor to:
cause, a transcranial magnetic stimulation (TMS) device, to generate a localized magnetic field waveform at a brain region; and
cause, an acoustic controller, to generate an audio waveform having at least one of rhythm, harmonic element, or melodic element synchronized to the localized magnetic field waveform.

16. The computer-readable medium of claim 15, wherein the instructions further causes the processor to:
receive a TMS waveform file; and
generate a TMS waveform to be amplified and subsequently applied to one or more TMS coils to generate the localized magnetic field waveform.

17. The computer-readable medium of claim 16, wherein the instructions further causes the processor to:
adjust a phase offset of the TMS waveform to synchronize the TMS waveform with the audio waveform.

18. The computer-readable medium of claim 17, wherein the instructions further causes the processor to:
adjust a phase offset of the audio waveform to synchronize the audio waveform with the TMS waveform.

19. The computer-readable medium of claim 15, wherein the brain region is the motor cortex region of the brain.
